(19) United States
(12) Patent Application Publication
(10) Pub. No.: US 2026/0263216 A1
(43) Pub. Date: Sep. 10, 2026

(54) LIMITED EXPANSION HEART VALVES

(71) Applicant: Edwards Lifesciences Corporation, Irvine, CA (US)

(72) Inventors: Derrick Johnson, Orange, CA (US); Michael C. Murad, Lake Mathews, CA (US); Steven M. Ford, Laguna Beach, CA (US); Rodolfo Rodriguez, San Luis Obispo, CA (US)

(21) Appl. No.: 19/665,491

(22) Filed: May 1, 2026

Related U.S. Application Data
(63) Continuation of application No. 17/487,232, filed on Sep. 28, 2021, now Pat. No. 12,629,251.
Application No. 17/487,232 is a continuation of application No. 16/721,727, filed on Dec. 19, 2019, now Pat. No. 11,135,057.
Application No. 16/721,727 is a continuation of application No. PCT/US2018/038527, filed on Jun. 20, 2018.
(60) Provisional application No. 62/523,157, filed on Jun. 21, 2017.

(51) Int. Cl. A61F 2/24

(52) U.S. Cl. CPC A61F 2/2418; A61F 2/24; A61F 2230/006

(57) ABSTRACT

A prosthetic heart valve configured to replace a native heart valve and having a support frame configured to be reshaped into an expanded form in order to receive and/or support an expandable prosthetic heart valve therein. A dual-wireform support frame including an upper and a lower wireform permits expansion of the valve by one or two valve sizes, for example, with a 2-mm gap between each valve size. The lower wireform has a relatively shallow undulation so that it may stretch apart by a small amount and then prevent further expansion of the valve.

### CROSS-REFERENCE TO RELATED APPLICATIONS

This application is a continuation of U.S. Patent Application No. 17/487,232, filed September 28, 2021, which is a continuation of U.S. Patent Application No. 16/721,727, filed December 19, 2019, now U.S. Patent No. 11,135,057, which is a continuation of International Patent Application No. PCT/US2018/ 038527, filed June 20, 2018, which claims the benefit of U.S. Patent Application No. 62/523157, filed June 21, 2017, the entire disclosures all of which are incorporated by reference for all purposes.

### BACKGROUND

The present disclosure relates to a heart valve for heart valve replacement, and more particularly to modifications to the construction of a surgical heart valve to enable receiving an expandable prosthetic heart valve therein and to expand to a limited degree.

The heart is a hollow muscular organ having four pumping chambers separated by four heart valves: aortic, mitral (or bicuspid), tricuspid, and pulmonary. Each heart valve is comprised of a dense fibrous ring known as the annulus, and leaflets or cusps attached to the annulus.

Heart valve disease is a widespread condition in which one or more of the valves of the heart fails to function properly. In a traditional valve replacement operation, the damaged leaflets are typically excised and the annulus sculpted to receive a replacement prosthetic valve.

In tissue-type valves, a whole xenograft valve (e.g., porcine) or a plurality of xenograft leaflets (e.g., bovine pericardium) can provide fluid occluding surfaces. Synthetic leaflets have been proposed, and thus the term “flexible leaflet valve” refers to both natural and artificial “tissue-type” valves. In a typical tissue-type valve, two or more flexible leaflets are mounted within a peripheral support structure that usually includes posts or commissures extending in the outflow direction to mimic natural fibrous commissures in the native annulus. The metallic or polymeric “support frame,” sometimes called a “wireform” or “stent,” has a plurality (typically three) of large radius cusps supporting the cusp region of the flexible leaflets (e.g., either a whole xenograft valve or three separate leaflets). The ends of each pair of adjacent cusps converge somewhat asymptotically to form upstanding commissures that terminate in tips, each extending in the opposite direction as the arcuate cusps and having a relatively smaller radius. Components of the valve are usually assembled with one or more biocompatible fabric (e.g., DACRON® polyethylene terephthalate) coverings, and a fabric-covered sewing ring is provided on the inflow end of the peripheral support structure.

Sometimes the need for complete valve replacement may arise after a patient has already had an earlier valve replacement for the same valve. For example, a prosthetic heart valve that was successfully implanted to replace a native valve may itself suffer damage and/or wear and tear many years after initially being implanted. Implanting a new prosthetic heart valve directly within a previously-implanted prosthetic heart valve (a so-called valve-in-valve procedure) may be impractical since traditional prosthetic heart valves may not be configured to easily receive such a valve-within-a-valve implantation in a manner which provides secure seating for the new valve while also having a large enough annulus within the new valve to support proper blood flow therethrough.

Some attention has been paid to the problem of implanting a new valve within an old valve. In particular, the following disclose various solutions for valve-in-valve systems: U.S. Patent Application Publication No. 2010/0076548 A1, filed September 19, 2008; U.S. Patent No. 8,613,765, filed July 7, 2011; and International Patent Application Publication No. WO 2012/018779, filed August 2, 2011. The entire disclosures of these publications are expressly incorporated herein by reference. Typically, the originally implanted heart valve is subjected to an outward dilatory force such as with an expanding balloon, until it expands to permit introduction of a new expandable valve within its orifice. The outward dilatory force from within the heart valve is typically substantially larger than forces associated with normal physiological cycling. The expansion may be done simultaneously with the new valve implantation.

### SUMMARY

Despite certain advances in valve-in-valve technology, there remains a need for a prosthetic heart valve that facilitates valve-in-valve procedures and simplifies manufacturing techniques.

Some embodiments provide a prosthetic heart valve configured to receive an expandable prosthetic heart valve, such as a catheter-deployed (transcatheter) prosthetic heart valve, therein. The prosthetic heart valve replaces a native heart valve and has a support frame configured to be reshaped into an expanded form in order to receive and/or support the expandable prosthetic heart valve therein. A dual-wireform support frame including an upper and a lower wireform permits expansion of the valve by one or two valve sizes, for example, with a 2-mm gap between each valve size. The expansion occurs upon application of an outward dilatory force from within the heart valve substantially larger than forces associated with normal physiological cycling. The lower wireform has a relatively shallow undulation so that it may stretch apart by a small amount and then prevent further expansion of the valve.

In a first aspect, the present application discloses a prosthetic heart valve adapted for post-implant expansion and having an inflow end and an outflow end. An upper wireform undulates around a central axis with three upstanding commissure posts extending in an outflow direction alternating with three arcuate inflow cusps, and a fabric covering around the entire upper wireform. A lower wireform undulates around the central axis with three truncated peaks extending in an outflow direction alternating with three arcuate inflow cusp sections, with a fabric covering around the entire lower wireform. The lower wireform is positioned axially below the upper wireform with the three truncated peaks being aligned under three upstanding commissure posts of the upper wireform, and wherein the truncated peaks have an axial height of between about 10–30% of the commissure posts. Three flexible leaflets having outer arcuate cusp edges attach between the inflow cusps of the upper wireform and the inflow cusp sections of the lower wireform. Outer tabs of the leaflets extend outward between the commissure posts of the upper wireform and the truncated peaks of the lower wireform and are secured to the fabric covering around the upper wireform, the flexible leaflets being configured to ensure one-way blood flow through the heart valve. The inflow cusps of the upper wireform and the inflow cusp sections of the lower wireform together define an implant circumference having a first diameter, wherein the upper and lower wireforms permit expansion of the heart valve from the first diameter to a second diameter no greater than 3 mm larger than the first diameter upon application of an outward dilatory force from within the heart valve substantially larger than forces associated with normal physiological cycling. Finally, the lower wireform has a shallow undulating shape that flattens out and prevents expansion of the heart valve beyond the second diameter

The prosthetic heart valve of the first aspect may further include three fabric-covered inserts located above the truncated peaks of the lower wireform that extend upward radially outward of the commissure posts of the upper wireform, the leaflet tabs being also secured to the inserts. Preferably, a lower end of each insert has an inverted Y-shape that closely matches a shape of the truncated peaks of the lower wireform.

The prosthetic heart valve of the first aspect may further include an annular sealing ring disposed outward of the inflow cusp sections of the lower wireform and being secured thereto, the annular sealing ring being suture permeable. In one embodiment, the lower wireform is embedded within the sealing ring.

The lower wireform may comprises a solid wire or a braided cable.

The prosthetic heart valve of the first aspect may further include an expandable frame attached to an inflow end of the heart valve and projecting therefrom in the inflow direction, the expandable frame having an upper undulating strut that extends around an entire periphery thereof and a plurality of lower struts. The undulating strut has a shape that closely follows the shape of the undulating lower wireform, wherein there are no lower struts below three peaks of the undulating strut to permit flattening out of the undulating strut upon application of an outward dilatory force from within the heart valve substantially larger than forces associated with normal physiological cycling.

In a second aspect, a prosthetic heart valve adapted for post-implant expansion and having an inflow end and an outflow end, comprises an upper wireform undulating around a central axis with three upstanding commissure posts extending in an outflow direction alternating with three arcuate inflow cusps, and a fabric covering around the entire upper wireform. An annular sealing ring is disposed outward of the inflow cusps of the upper wireform and is secured thereto, the annular sealing ring being suture permeable. A braided cable undulates around the central axis with three truncated peaks extending in an outflow direction alternating with three arcuate inflow cusp sections, the braided cable being embedded within the sealing ring and the three truncated peaks being aligned under three upstanding commissure posts. Three flexible leaflets having outer arcuate cusp edges attach between the inflow cusps of the upper wireform and the sealing ring. Outer tabs of the leaflets extend outward between the commissure posts of the upper wireform and are secured to the fabric covering around the upper wireform, the flexible leaflets being configured to ensure one-way blood flow through the heart valve. The inflow cusps of the upper wireform and the inflow cusp sections of the braided cable together define an implant circumference having a first diameter, wherein the upper wireform and braided cable permit expansion of the heart valve from the first diameter to a second diameter no greater than 3 mm larger than the first diameter upon application of an outward dilatory force from within the heart valve substantially larger than forces associated with normal physiological cycling. Finally, the braided cable has a shallow undulating shape that flattens out and prevents expansion of the heart valve beyond the second diameter

The prosthetic heart valve of the second aspect may further include three fabric-covered inserts located above the truncated peaks of the braided cable that extend upward radially outward of the commissure posts of the upper wireform, the leaflet tabs being also secured to the inserts. Lower ends of each insert may have an inverted Y-shape that closely matches a shape of the truncated peaks of the braided cable.

The braided cable may be joined together at free ends at a weld in one of the cusp sections, or at a crimp at one of the truncated peaks.

The prosthetic heart valve of the second aspect may further include an expandable frame attached to an inflow end of the heart valve and projecting therefrom in the inflow direction, the expandable frame having an upper undulating strut that extends around an entire periphery thereof and a plurality of lower struts. The undulating strut has a shape that closely follows the shape of the undulating lower wireform, wherein there are no lower struts below three peaks of the undulating strut to permit flattening out of the undulating strut upon application of an outward dilatory force from within the heart valve substantially larger than forces associated with normal physiological cycling.

Other features and advantages will become apparent from the following detailed description, taken in conjunction with the accompanying drawings that illustrate, by way of example, certain principles and examples.

### BRIEF DESCRIPTION OF THE DRAWINGS

FIGS. 1A–1E are a number of views of an exemplary prosthetic heart valve of the present invention having a dual wireform construction including an expansion limiting suture in place of inner structural bands;

FIG. 2A is an elevational view of an upper wireform, FIG. 2B is an elevational view of a lower wireform, and FIG. 2C is a schematic view showing the upper and lower wireforms in the positions they assume when assembled within a heart valve;

FIG. 3 is a view of the dual wireform assembly showing exemplary dimensions;

FIG. 4 is a view of the dual wireform assembly after expansion and showing altered dimensions;

FIG. 5 is a sectional view through a heart valve having a dual wireform assembly where the lower wireform is a braided cable;

FIGS. 6A and 6B are perspective views of two different braided cable wireforms;

FIG. 7A is a partially cutaway view of another exemplary prosthetic heart valve having an expandable frame attached to an inflow end, and FIG. 7B is an elevational view of the heart valve where only the expandable frame is shown in solid lines;

FIG. 8A is a perspective view of the expandable frame isolated from the heart valve; and

FIG. 8B is a perspective view of the expandable frame after expansion.

### DETAILED DESCRIPTION

The prosthetic heart valves disclosed herein include a prosthetic valve member constructed similarly to embodiments of some commercially available surgical valves, with a relatively stable diameter that is not intended to be compressed or expanded during delivery and after implant when functioning as a one-way valve. The prosthetic heart valves described herein each include an internal (meaning incorporated into the valve member itself as opposed to being a supplemental element) structural stent or frame that is generally tubular in shape and that defines a flow orifice area through which blood flows from an inflow end to an outflow end. Alternatively, the shape of the internal stent can be oval, elliptical, D-shaped, irregular, or any other desired and functional shape. The valves include flexible leaflets that selectively open and close to allow for one-way fluid flow therethrough.

The present application discloses specific modifications to existing surgical valves that enable manufacturers to rapidly produce a valve that accommodates valve-in-valve (ViV) procedures. Specifically, the present application contemplates modifying certain components within existing surgical valve designs to enable post-implant expansion, which not only converts any proven surgical valve design for use in a ViV procedure, but it also reduces design and manufacturing work. Consequently, components of one popular surgical valve are described below to illustrate certain modifications thereto.

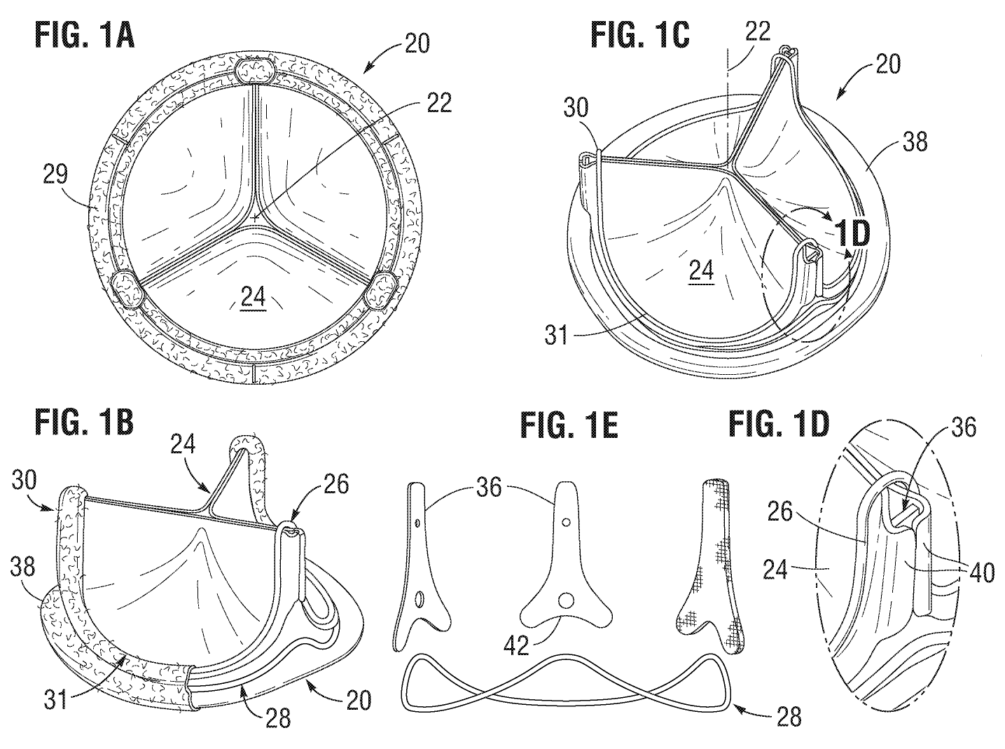

FIGS. 1A–1E are various views of an exemplary surgical prosthetic heart valve 20 oriented around a flow axis 22. The heart valve 20 comprises a plurality (typically three) of flexible leaflets 24 supported partly by an undulating upper wireform 26 as well as by a lower wireform 28. The upper wireform 26 and lower wireform 28 are visible in the figures, but are normally separately covered with a polyester fabric to facilitate assembly and reduce direct blood exposure after implant. The directions up and down are aligned along the flow axis 22 and generally correspond to flow directions, with the blood flowing up along the axis past the leaflets 24 in an outflow direction when the heart valve 20 is implanted.

Certain characteristics of the prosthetic heart valve 20 are common to a number of different prosthetic heart valves, such as pericardial heart valves manufactured by Edwards Lifesciences of Irvine, CA. For example, the Edwards PERIMOUNT® heart valves that utilize pericardial leaflets 24 features a leaflet-supporting wireform such as the upper wireform 26, but also has an inner stent comprising a relatively non-expandable circular band structure. The exemplary heart valve 20 disclosed herein improves on the PERIMOUNT® heart valves by avoiding inner support structure which inhibits post-implant expansion.

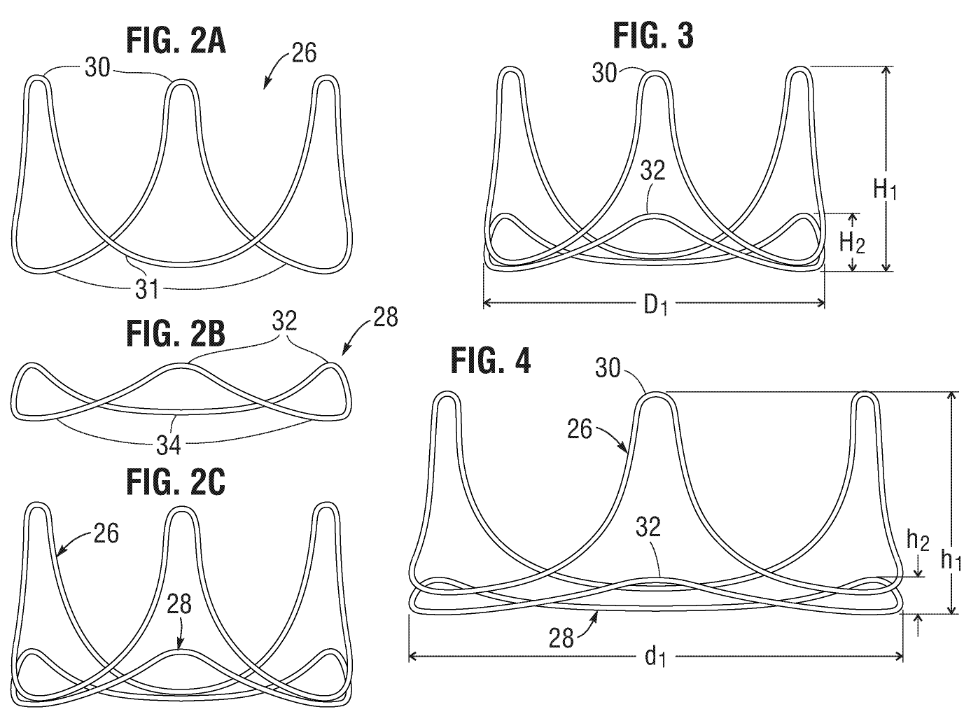

FIG. 2A is an elevational view of the upper wireform 26, FIG. 2B is an elevational view of a lower wireform 28, and FIG. 2C is a schematic view showing the upper and lower wireforms in the approximate positions they assume when assembled within the heart valve 20. The upper wireform 26 may be formed from a suitably elastic metal, such as a Co-Cr-Ni alloy like ELGILOY® alloy. The upper wireform 26 has a continuous undulating wire-like structure with (preferably) three upstanding commissure posts 30 in between three downwardly curved valleys typically termed cusps 31, as best seen in FIG. 1C. The wireform 26 forms narrow inverted “U” shapes at the commissure posts 30 that project in the outflow direction and define the farthest extent of the valve in that direction aside from fabric covering. This undulating band shape is useful for prosthetic aortic heart valves, which typically have three leaflets joined at their adjacent edges at the upstanding commissure posts 30. Of course, the heart valves disclosed herein may be utilized in other implant locations, such as the pulmonary, mitral, or tricuspid annulus.

The lower wireform 28 is preferably metallic as well, but may be solid or a braided structure, as will be discussed. As seen in FIG. 1B, the lower wireform 28 has generally the same shape as the upper wireform 26 but with three truncated peaks 32 intermediate three cusp sections 34. The three cusp sections 34 closely parallel the cusps 31 of the upper wireform 26, but the truncated peaks 32 terminate well below the commissure posts 30.

In the illustrated embodiment, the peaks 32 of the lower wireform 28 are rotationally aligned with the commissure posts 30 of the upper wireform 26. In other embodiments, one or more of the peaks 32 is rotationally offset from the commissure posts 30. For example, in some embodiments, at least two peaks 32 are rotationally offset in the same direction. In some embodiments, at least a first peak is rotationally offset in an opposite direction as a second peak. In some embodiments, a first peak is rotationally offset by a different angular distance than a second peak.

Moreover, although the illustrated embodiment of the upper wireform 26 includes three commissure posts 30, in other embodiments, the upper wireform includes a different number of commissure posts, for example, two or four. In the illustrated embodiment, the number of peaks 32 on the lower wireform 28 matches the number of commissure posts 30 on the upper wireform 26: in this example, three of each. In other embodiments, the number of peaks is different than the number of commissure posts. For example, some embodiments include fewer peaks than commissure posts, for example, two peaks on a device with three commissure posts. Other embodiments include more peaks than commissures, for example, by replacing at least one of the peaks 32 with two peaks.

FIG. 3 is a view of the dual wireform assembly in a relaxed, unexpanded configuration showing exemplary dimensions. In a preferred embodiment, the truncated peaks 32 of the lower wireform 28 have an axial height H₂ of only about 10–30% of the axial height H₁ of commissure posts 30 of the upper wireform 26, and more preferably about 20%. The upper and lower wireforms 26, 28 define a circle of rotation at their inlet ends having a common diameter D₁, with the two wireforms axially stacked and the lower wireform just below the upper wireform. Typically, heart valves are available in labeled sizes from 19 to up to 33 mm in 2-mm increments (e.g., 19 mm, 21 mm, 23 mm…), and the diameter D₁ is between 19–33 mm, roughly corresponding to the labeled diameter of the finished valve 20. Other sizing schemes are also possible, for example, even millimeter sizing, and/or a sizing scheme implementing at least one different increment between sizes. The valves 20 disclosed herein have a functional size which equals the labeled size, whereas the valve becomes non-functional when expanded outward post-implant.

FIG. 4 is a view of the dual wireform assembly after expansion and showing altered dimensions d₁, h₁, h₂. Namely, the dimension or diameter d₁ widens or increases by up to about 2–3 mm, preferably closer to about 2 mm for smaller valves and about 3 mm for larger valves. Recent publications report a drastically higher probability of annular rupture upon expanding the native annulus by more than 20% by area, such as when expanding a prosthetic heart valve therein. In light of this information, it is desirable to ensure that an expandable surgical valve expands by less than about 20% by area in some embodiments. Thus, for example, for a 19-mm valve a 20% increase in area corresponds to an increase in diameter of about 2 mm.

In other embodiments, the upper and lower wireforms 26, 28 do not have a common diameter. For example, in some embodiments, the lower wireform has a larger diameter than the upper wireform. In some of these embodiments, such a configuration permits nesting the upper wireform within the lower wireform, thereby reducing the overall height (H₁ and h₁) of the device. In some of these embodiments, the final diameters (d₁ in FIG. 4) of the upper and lower wireforms is different, while in other embodiments, the final diameters are substantially the same.

The heights h₁, h₂ of the upper and lower wireforms 26, 28, respectively, decrease when the wireforms expand. Because of the relatively high commissure posts 30 of the upper wireform 26, and their large capacity to expand outward toward the cusps 31, the height h₁ decreases a smaller proportion of the original height H₁compared with h₂/H₂. However, since the lower wireform 28 has relatively shallower undulations between the peaks 32 and cusp sections 34 compared with the upper wireform 26, the reduced height h₂ is preferably less than about 50% of the original height H₂. More preferably, the lower wireform 28 flattens out to a great extent to more closely resemble a flat ring, thus presenting a relatively strong impediment to further expansion, such as with an expanding balloon during a valve-in-valve procedure. The expanded lower wireform 28 is shown with slight undulations, although it could be much flatter depending on the original height H₂ and the extent of expansion. Preferably the hoop strength of the lower wireform 28 increases to a magnitude sufficient to withstand balloon expansion from within after an expansion of between about 2–3 mm in diameter.

With reference back to FIG. 1D, further constructional details of the heart valve 20 include a plurality of inserts 36 which are located generally between the commissure posts 30 of the upper wireforms 26 and the peaks 32 of the lower wireforms 28 and help secure the leaflets 24 in place. One of the inserts 36 is shown covered with cloth in FIG. 1E. Additionally, a suture permeable sealing ring 38 surrounds the inlet end of the valve 20 and is used to secure the valve to the annulus. Typically, the sealing ring 38 comprises silicone, cloth or other such suture-permeable material, and is covered in fabric as seen in FIG. 1B.

Outer tabs 40 of adjacent leaflets 24 wrap around upper ends of commissure inserts 36 (preferably three) that project in an outflow direction along the flow axis 22. The commissure inserts 36 comprises elements separate from either the upper and lower wireforms 26, 28, and each has an inverted “Y” shape with a forked lower end 42 that generally conforms to a peak 30 of the lower wireform 28. Once covered in fabric, as illustrated for the one of the inserts shown in FIG. 1E, the inserts 36 are preferably positioned above the fabric-covered lower wireform 28 and secured to the leaflets 24 and fabric-covered upper wireform 26. Arcuate cusp edges of the leaflets 24 preferably extend between the cloth covered wireforms 26, 28 and are secured thereto with sutures.

Once assembled with the other valve components, the combination of the upper and lower wireforms 26, 28 presents a relatively dimensionally stable circumferential base to the valve 20, which is beneficial for typical surgical use. That is, primarily the lower wireform 28 provides good ring support to the cusp edges of the leaflets 24 and helps provide resistance to deformation of the valve during implantation. However, because of its undulating shape, the lower wireform 28 facilitates limited expansion of the valve 20.

During a valve-in-valve procedure, as the lower wireform 28 expands, the commissure posts 30 become spaced apart since the upper wireform 26 expands outward, which may lead to a loss of function of the valve 20. However, the valve becomes obsolete, having been replaced with a transcatheter valve, and so this loss of function is of no consequence. The wireform maintains the upstanding commissure posts of the expanded valve in roughly the same relative circumferential locations as when they were functional, which are intermediate the surrounding coronary ostia, and thus valve expansion will not end up blocking critical blood flow to the coronary arteries.

Another concept for limiting the expansion of prosthetic heart valves is shown in FIG. 5, which is a sectional view through an alternative heart valve 50 also having a dual wireform assembly where a lower wireform 52 is a braided cable. As before, the heart valve 50 has a cloth-covered upper wireform 54 and a plurality of leaflets 56 supported thereby. An outer sealing ring 58 includes a taller axial portion 60 at each of the commissure locations, which may be a molded silicone element or folded cloth or the like. Although not shown, commissure inserts such as those shown above at 36 may be utilized, and an outer cloth covering is not shown for clarity.

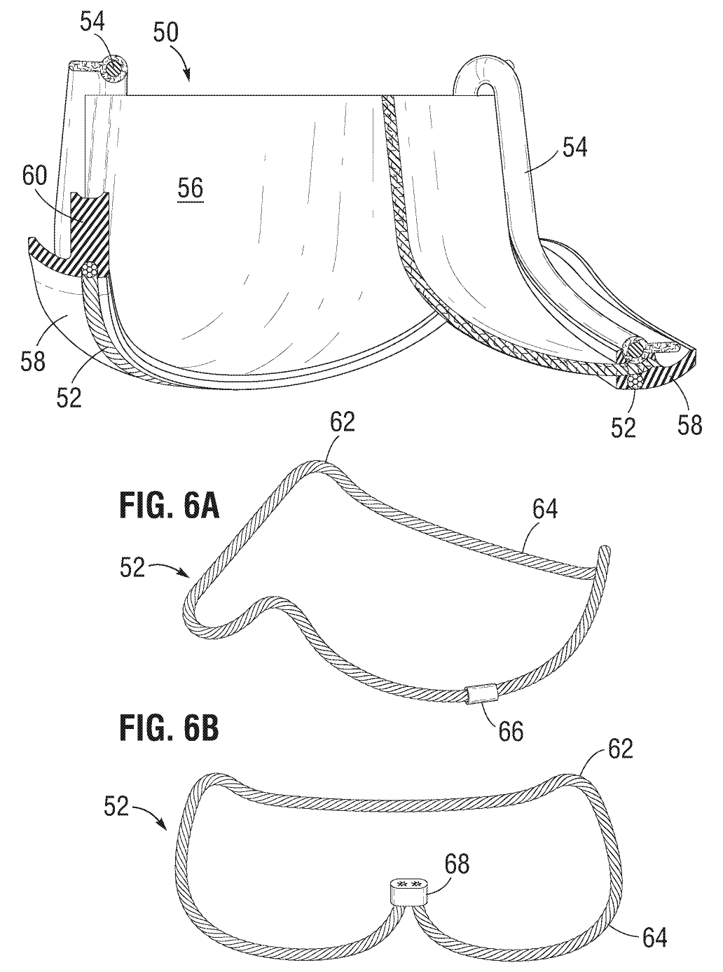

The lower wireform 52 is preferably shaped similarly to the lower wireform 28 described above, and is shown in two different embodiments in FIGS. 6A and 6B. Namely, the wireform 52 has an undulating shape with truncated peaks 62 in between arcuate cusp sections 64. The braided wireform 52 is preferably formed from an elongated braided cable or wire which is joined together at its free ends at either a weld 66 as seen in FIG. 6A, or at a crimp 68 such as seen in FIG. 6B. A weld 66 is typically used in the cusp sections 64, while a crimp 68 would be preferred at one of the peaks 62. Although not shown, the braided cable or wire is preferably held in the undulating shape as shown, such as with the use of a mandrel or other such manufacturing form, and heat set so that the shape is imparted to the cable. In a preferred embodiment, the braided wireform 52 is made of a plurality of braided strands of Nitinol that have been heat set. In this way, the wireform 52 provides a relatively stable peripheral base for the valve 50, but is also relatively flexible and permits post-implant expansion. In other embodiments, the braided wireform 52 comprises strands manufactured from another material, for example, stainless steel or cobalt-chromium. In other embodiments, the cable comprises a polymer, for example, ultra-high-molecular-weight polyethylene (UHMWPE, e.g., Spectra® (Honeywell, Morristown, New Jersey) or Dyneema® (Heerlen, Netherlands) UHMWPE)) or polyaramid (e.g., Kevlar® (DuPont, Wilmington, Delaware) or Twaron® (Teijin, Arnhem, Netherlands) aramid). Other embodiments of the cable comprise a composite including at least two of any of these materials. Some examples of the braided wireform 52 are manufactured in an annular shape, and consequently, do not include a weld or crimp. Examples of suitable manufacturing methods include weaving, knitting, or braiding.

In contrast to the lower wireform 28 described above, the braided wireform 52 is desirably embedded within the sealing ring 58, although the lower wireform 28 may also be embedded within the sealing ring. In one embodiment, the sealing ring 58 is a molded silicone element having the braided wireform 52 co-molded in an underside thereof. As mentioned, the assembly of the wireform 52 and sealing ring 58 may be covered with fabric and then joined to the upper wireform 54 and leaflets 56 via sutures. In FIG. 5, the cable 58 is disposed directly below the wireform 54. In other embodiments, the cable and wireform are radially offset. For example, as discussed above in connection with the lower wireform 28, the wireform 52 can nest within a cable with a larger diameter.

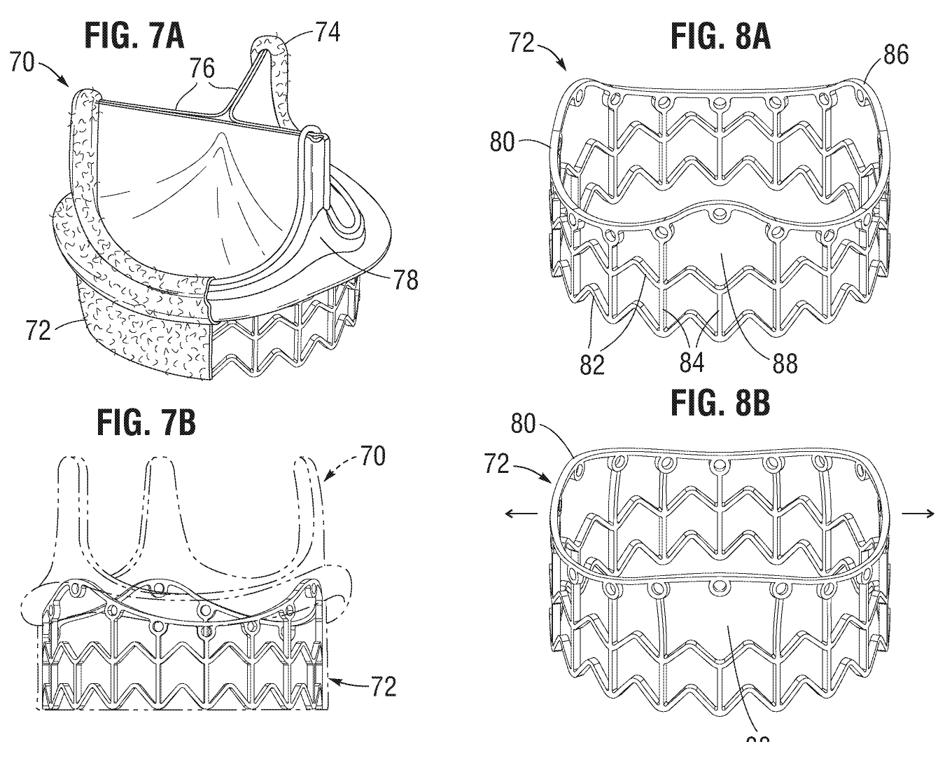

FIG. 7A is a partially cutaway view of another exemplary prosthetic heart valve 70 having an expandable frame 72 attached to an inflow end, and FIG. 7B is an elevational view of the heart valve where only the expandable frame is shown in solid lines. As described above, the heart valve 70 includes an undulating wireform 74 that supports a plurality of flexible leaflets 76. Element number 78 refers to an inner support member which is adapted for post-implant expansion. That is, the support member 78 may comprise the lower wireform 28, as described above, or may be a band structure which has at least one section adapted to expand from use of a dilatation balloon.

The addition of the expandable frame 72 creates a “hybrid” type of prosthetic heart valve in that the upper portion is constructed similar to a surgical valve, while the lower frame structure 72 is expandable to help in anchoring the valve in place. One specific commercial prosthetic heart valve that is constructed in this manner is one which is sold in conjunction with the Edwards Intuity® valve system from Edwards Lifesciences of Irvine, CA. The Edwards Intuity® valve system comprises a “hybrid” valve incorporating essentially a surgical Perimount® valve, albeit one that is modified for post-implant expansion, and a stainless steel lower frame structure or skirt stent.

FIG. 8A is a perspective view of the expandable frame 72 isolated from the heart valve 70, and FIG. 8B is a perspective view of the expandable frame after expansion. The frame 72 includes an upper undulating strut 80 that extends around the entire periphery of the frame and above a plurality of generally V-shaped circumferential struts 82 extending between axial struts 84. The undulating strut 80 includes three peaks 86 that generally conform to the undulating shape of the inflow end of the heart valve 70, as best seen in FIG. 7B. In other words, the three peaks 86 correspond to the three commissures 74 of the valve. An absence of the vertical struts 84 immediately below each of the three peaks 86 creates a space or void 88. Due to the upper curvature of the peaks 86, this permits the undulating strut 80 to expand outward such as seen in FIG. 8B upon application of a dilatory force within the hybrid prosthetic valve.

In one embodiment, the aforementioned inner support member 78 may be omitted completely from the prosthetic valve 70 with the undulating strut 80 providing support to the base of the valve and the leaflets. For example, the undulating strut 80 may be positioned approximately the same place as the braided wireform 52 seen in FIG. 5. To prevent premature expansion of the undulating strut 80 at the time of implant of the valve 70, a biodegradable band may be assembled around the inflow end of the surgical valve 70. Such a biodegradable band is seen in FIG. 15 of US 9,375,310 to Chung, et al., the contents of which are expressly incorporated herein by reference, and serves to prevent expansion of the upper end of the frame 72 at the time of initial implant of the valve 70. Subsequently, years later, if the valve 70 malfunctions, the biodegradable band has dissolved and a dilatation balloon can easily expand the undulating strut 80.

While certain principles have been described with reference to particular embodiments, it will understood that various changes and additional variations may be made and equivalents may be substituted for elements thereof without departing from the scope of the disclosure. In addition, many modifications may be made to adapt a particular situation or device to the teachings without departing from the essential scope thereof. Therefore, it is intended that the disclosure not be limited to the particular embodiments disclosed herein, but will include all embodiments falling within the scope of the appended claims.

What is claimed is:

1. A prosthetic heart valve adapted for post-implant expansion and having an inflow end and an outflow end, comprising: a metallic or polymeric support frame surrounding a central axis and having an undulating shape around an annular periphery thereof with three upstanding commissure posts spaced apart 120° and extending in an outflow direction alternating with three arcuate inflow cusps each spanning 120°, and the commissure posts and inflow cusps together define the annular periphery, and a fabric covering around the support frame; three flexible leaflets attached within the support frame and being secured to the fabric covering, each leaflet having an outer arcuate cusp edge aligned with one of the inflow cusps, the flexible leaflets being configured to ensure one-way blood flow through the heart valve; a wireform undulating with three peaks extending in an outflow direction alternating with three arcuate inflow cusp sections, the wireform being positioned co-axially with respect to the support frame with the three peaks being aligned with the three commissure posts, and wherein the peaks have an axial height of between about 10–30% of the commissure posts of the support frame; and wherein the cusp sections of the wireform define an implant circumference having a first diameter within the wireform, and wherein the wireform permits expansion of the heart valve from the first diameter to a second diameter larger than the first diameter upon application of an outward dilatory force from within the heart valve substantially larger than forces associated with normal physiological cycling.

2. The prosthetic heart valve of claim 1, further including an annular sealing ring disposed outward of the cusp sections of the wireform and being secured thereto, the annular sealing ring being suture permeable.

3. The prosthetic heart valve of claim 2, wherein the wireform is embedded within the sealing ring.

4. The prosthetic heart valve of claim 1, wherein the wireform comprises a solid wire.

5. The prosthetic heart valve of claim 1, wherein the cable comprises a polymer.

6. The prosthetic heart valve of claim 5, wherein the polymer is selected from the group consisting of: ultra-high-molecular-weight polyethylene and polyaramid.

7. The prosthetic heart valve of claim 1, wherein the wireform comprises a braided cable.

8. The prosthetic heart valve of claim 7, wherein the braided cable is made of braided strands of a material selected from the group consisting of: Nitinol, stainless steel, and cobalt-chromium, or a combination of two of these materials.

9. The prosthetic heart valve of claim 7, wherein the braided cable is made of braided strands of a polymer.

10. The prosthetic heart valve of claim 1, wherein the wireform comprises a lower wireform and the support frame further includes an upper wireform undulating around the central axis with three upstanding commissure posts extending in an outflow direction alternating with three arcuate inflow cusps, the arcuate inflow cusps being aligned with the cusp sections of the lower wireform, and wherein the lower wireform flattens out and prevents expansion of the heart valve beyond the second diameter.

11. The prosthetic heart valve of claim 1, wherein the wireform is disposed directly below the support frame.

12. The prosthetic heart valve of claim 1, wherein the wireform is radially offset with respect to the support frame.

13. A prosthetic heart valve adapted for post-implant expansion and having an inflow end and an outflow end, comprising: a metallic or polymeric support frame surrounding a central axis and having an undulating shape around an annular periphery thereof with three upstanding commissure posts spaced apart 120° and extending in an outflow direction alternating with three arcuate inflow cusps each spanning 120°, and the commissure posts and inflow cusps together define the annular periphery, and a fabric covering around the support frame; three flexible leaflets having outer arcuate cusp edges attached within the support frame and being secured to the fabric covering, the flexible leaflets being configured to ensure one-way blood flow through the heart valve; a suture permeable annular sealing ring defining an outer periphery of the heart valve and attached to the support frame at an inflow end thereof, the sealing ring being a molded element; and a wireform embedded within the molded sealing ring and having a shallow undulating shape around an annular periphery thereof with three peaks spaced apart 120° and extending in an outflow direction alternating with three arcuate inflow cusp sections each spanning 120°, the three peaks being aligned with the three commissure posts and the peaks and cusp sections together define the annular periphery, wherein the peaks have an axial height of between about 10–30% of the commissure posts of the support frame; wherein the cusp sections of the wireform define an implant circumference having a first diameter within the wireform, and wherein the wireform permits expansion of the heart valve from the first diameter to a second diameter larger than the first diameter upon application of an outward dilatory force from within the heart valve substantially larger than forces associated with normal physiological cycling.

14. The prosthetic heart valve of claim 13, wherein the wireform comprises a braided cable.

15. The prosthetic heart valve of claim 14, wherein the braided cable is joined together at free ends at a weld in one of the cusp sections.

16. The prosthetic heart valve of claim 14, wherein the braided cable is joined together at free ends at a crimp at one of the peaks.

17. The prosthetic heart valve of claim 14, wherein the braided cable is made of braided strands of a material selected from the group consisting of: Nitinol, stainless steel, and cobalt-chromium, or a combination of two of these materials.

18. The prosthetic heart valve of claim 14, wherein the braided cable is made of braided strands of a polymer.

19. The prosthetic heart valve of claim 13, wherein the wireform comprises a polymer.

20. The prosthetic heart valve of claim 19, wherein the polymer is selected from the group consisting of: ultra-high-molecular-weight polyethylene and polyaramid.

21. The prosthetic heart valve of claim 13, wherein the sealing ring is silicone and the wireform is co-molded in an underside thereof.

22. The prosthetic heart valve of claim 13, wherein the shallow undulating shape of the wireform flattens out and prevents expansion of the heart valve beyond the second diameter.

23. The prosthetic heart valve of claim 13, wherein the wireform is disposed directly below the support frame.

24. The prosthetic heart valve of claim 13, wherein the wireform is radially offset with respect to the support frame.
